(19) United States
(12) Patent Application Publication
(10) Pub. No.: US 2011/0150974 A1
(43) Pub. Date: Jun. 23, 2011

(54) Agent For Oral Mucosal Administration

(75) Inventors: Noriko Nakajima, Saitama (JP); Norihiro Shinkai, Saitama (JP); Hitoshi Yamauchi, Saitama (JP); Masahiko Kikuchi, Tokyo (JP)

(73) Assignees: DAIICHI PHARMACEUTICAL CO., LTD., Tokyo (JP); SAITAMA DAIICHI PHARMACEUTICAL CO., LTD., Saitama (JP)

(21) Appl. No.: 11/573,209

(22) PCT Filed: Aug. 4, 2005
(86) PCT No.: PCT/JP05/14274

§ 371 Date: Nov. 5, 2007

(30) Foreign Application Priority Data
Aug. 6, 2004 (JP) 2004-230054

(51) Int. Cl. A61K 31/4747; A61K 9/24; A61L 15/16

(52) U.S. Cl. 424/448; 514/278; 424/472

(57) ABSTRACT

A medicament used for prophylactic and/or therapeutic treatment of xerostomia, which is in the form for oral mucosal administration comprising a spirooxathiolane quinuclidine derivative represented by the following general formula (I) or an acid addition salt thereof:

[Formula 1]

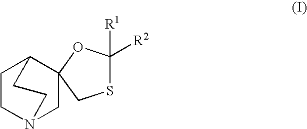

(wherein R¹ and R² may be the same or different, and independently represent a hydrogen atom, an alkyl group, a cyclopentyl group, a cyclohexyl group, a monoaryl- or diaryl-substituted methylol group, or an aryl-substituted alkyl group) as an active ingredient.

### TECHNICAL FIELD

The present invention relates to an agent for oral mucosal administration comprising a spirooxathiolane quinuclidine derivative or an acid addition salt thereof as an active ingredient.

### BACKGROUND ART

As agents for oral mucosal administration for the treatment of xerostomia, moisturizing agents have conventionally been used, such as artificial saliva used as a saliva substitute, or candy-like substances and chewing gums that promote saliva secretion by utilizing stimulation with acids such as citric acid and vitamin C. However, none of these drugs achieves sufficient effects, and therefore, development of drugs having superior effectiveness for xerostomia has been desired.

In recent years, oral preparations using a hydrate of 2-methylspiro(1,3-oxathiolane-5,3′)quinuclidine hydrochloric acid addition salt (also referred to as “cevimeline hydrochloride hydrate”) have come to be used as therapeutic agents for xerostomia caused by Sjogren's syndrome, and their saliva secretion promoting effect has been confirmed (Japanese Patent Unexamined Patent Publication (KOKAI) Nos. 61-280497 and 06-024981). However, oral preparations using cevimeline hydrochloride hydrate may cause symptoms such as nausea, abdominal pain, and vomiting as adverse reactions in the digestive system. It is presumed that these adverse reactions are caused by the active ingredient distributed at high concentrations in tissues of the stomach, small intestine and the like, which are the absorption sites after oral administration of cevimeline hydrochloride hydrate. However, the onset mechanism of the adverse reactions remains unrevealed. If the aforementioned adverse reactions caused by a preparation using a cevimeline hydrochloride hydrate can be avoided, high usefulness thereof as a therapeutic agent for xerostomia can be expected. However, such a preparation has not yet been provided so far.

Patent document 1: Japanese Patent Unexamined Patent Publication No. 61-280497
Patent document 2: Japanese Patent Unexamined Patent Publication No. 06-024981

### DISCLOSURE OF THE INVENTION

### Object to be Achieved by the Invention

An object of the present invention is to provide a medicament for prophylactic and/or therapeutic treatment of xerostomia. More specifically, the object of the present invention is to provide a medicament for prophylactic and/or therapeutic treatment of xerostomia, which can quickly exhibit a superior saliva secretion promoting effect and can reduce or eliminate the adverse reactions in the digestive organs.

### Means for Achieving the Object

The inventors of the present invention conducted various researches to achieve the foregoing object. As a result, they found that the foregoing object was successfully achieved by providing a medicament in a form for oral mucosal administration containing a spirooxathiolane quinuclidine derivative represented by the following general formula (I) or an acid addition salt thereof as an active ingredient. More specifically, they found that a medicament in the form for oral mucosal administration comprising the aforementioned active ingredient had superior absorbability and distribution property to the salivary glands and successfully exhibited a superior saliva secretion promoting effect without causing adverse reactions in the digestive organs. Further, the inventors of the present invention also found that, by using a particular pH condition or a hydrophilic solvent in the aforementioned medicament, the medicament having higher oral mucosal absorbability was successfully provided, and that the medicament exhibited higher prophylactic and/or therapeutic effect as an oral mucosa adhesive patch. The present invention was accomplished on the basis of the aforementioned findings.

The present invention thus provides a medicament in a form for oral mucosal administration which comprises a spirooxathiolane quinuclidine derivative represented by the following general formula (I):

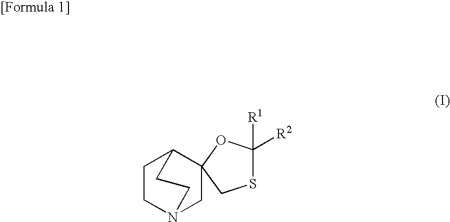

(wherein R¹ and R² may be the same or different, and independently represent a hydrogen atom, an alkyl group, a cyclopentyl group, a cyclohexyl group, a monoaryl- or diaryl-substituted methylol group, or an aryl-substituted alkyl group) or an acid addition salt thereof as an active ingredient. This medicament is used as a medicament for prophylactic and/or therapeutic treatment of xerostomia.

According to preferred embodiments of the aforementioned invention, provided are the aforementioned medicament, wherein the spirooxathiolane quinuclidine derivative is 2-methylspiro(1,3-oxathiolane-5,3′-quinuclidine); the aforementioned medicament, wherein the spirooxathiolane quinuclidine derivative is cis 2-methylspiro(1,3-oxathiolane-5,3′-quinuclidine); the aforementioned medicament, wherein the active ingredient is 2-methylspiro(1,3-oxathiolane-5,3′-quinuclidine) hydrochloride or a hydrate thereof, the aforementioned medicament, wherein the active ingredient is cis-2-methylspiro(1,3-oxathiolane-5,3′-quinuclidine) hydrochloride or a hydrate thereof, and the aforementioned medicament, wherein the active ingredient is cis-2-methylspiro(1,3-oxathiolane-5,3′-quinuclidine) monohydrochloride hemihydrate.

According to further preferred embodiments of the present invention, provided are the aforementioned medicament, which is in the form of a pharmaceutical composition containing a water-soluble or swellable polymer and/or a hydrophilic solvent; the aforementioned medicament, wherein the water-soluble or swellable polymer consists of one or more kinds of polymers selected from the group consisting of methylcellulose, ethylcellulose, hydroxyethylcellulose, hydroxymethylethylcellulose, hydroxypropylcellulose, hydroxypropylmethylcellulose, carboxymethylcellulose sodium, xanthan gum, karaya gum, gum arabic, tragacanth gum, cyamoposis gum, gelatin, polyvinylpyrrolidone, polyvinyl alcohol, polyacrylic acid or a metal salt thereof, a carboxyvinyl polymer, an alginic acid salt, propylene glycol alginate, pullulan, a lower alkyl vinyl ether/maleic anhydride copolymer and a derivative thereof; the aforementioned medicament, wherein the water-soluble or swellable polymer consists of one or more kinds of polymers selected from the group consisting of methylcellulose, ethylcellulose, hydroxyethylcellulose, hydroxymethylethylcellulose, hydroxypropylcellulose, hydroxypropylmethylcellulose and carboxymethylcellulose sodium; the aforementioned medicament, wherein the hydrophilic solvent consists of one or more kinds of solvents selected from the group consisting of monoethanolamine, diethanolamine, triethanolamine, diisopropanolamine, triisopropanolamine, ethanol, isopropanol, glycerin, ethylene glycol, propylene glycol, 1,3-butylene glycol, N-methyl-2-pyrrolidone, polyethylene glycol 300, polyethylene glycol 400, triethyl citrate, a glycerin fatty acid ester, polysorbate 20, polysorbate 40, polysorbate 60 and polysorbate 80; and the aforementioned medicament, wherein the hydrophilic solvent consists of one or more kinds of solvents selected from the group consisting of monoethanolamine, diethanolamine, triethanolamine, diisopropanolamine and triisopropanolamine.

According to further preferred embodiments, provided are the aforementioned medicament, wherein pH of the pharmaceutical composition is in the range of 6.5 to 9; the aforementioned medicament, which is in the form of a pharmaceutical composition of patch, sublingual tablet, buccal tablet, solution, gargle, spray, aerosol, ointment, jelly or film; the aforementioned medicament, which is in the form of an oral mucosal patch comprising (1) a water-insoluble support layer and (2) an adhesive layer having adhesion to mucosa laminated on the aforementioned support layer; the aforementioned medicament, wherein the water-insoluble support layer contains one or more kinds of substances selected from the group consisting of ethylcellulose, hydroxyethylcellulose, hydroxypropylmethylcellulose phthalate, hydroxypropylmethylcellulose acetate succinate, cellulose acetate phthalate, shellac, polyisobutylene and polyisoprene, and the mucosa adhesive layer contains a water-soluble or swellable polymer that develops adhesion with moisture in the oral cavity, and a hydrophilic solvent.

From another aspect of the present invention, there are also provided use of a spirooxathiolane quinuclidine derivative represented by the aforementioned general formula (I) or an acid addition salt for manufacture of the aforementioned medicament in the form for oral mucosal administration; and a method for prophylactic and/or therapeutic treatment of xerostomia comprising the step of administering a pharmaceutical composition comprising a spirooxathiolane quinuclidine derivative represented by the aforementioned general formula (I) or an acid addition salt thereof to the oral mucosa.

### EFFECT OF THE INVENTION

The medicament of the present invention allows very quick distribution of the spirooxathiolane quinuclidine derivative or an acid addition salt thereof as the active ingredient from the oral mucosa into the salivary glands to exhibit a superior saliva secretion promoting effect from just after the administration. Further, with the medicament of the present invention, absorption of the active ingredient is attained from the oral mucosa, thus distribution of the active ingredient into the gastrointestinal tract can be remarkably reduced. Therefore, the developments of adverse reactions in the digestive organs, which are problems of conventional oral agents, can be reduced or eliminated.

### BRIEF DESCRIPTION OF THE DRAWINGS

[FIG. 1] A schematic view of an oral mucosa adhesive patch comprising cevimeline hydrochloride hydrate as an active ingredient.

[FIG. 2] A graph showing effects of additives on oral mucosal absorption of cevimeline hydrochloride hydrate. In the graph, “TEA” represents triethanolamine, and HCl represents hydrochloric acid.

[FIG. 3] A graph showing effects of agents for oral mucosal administration comprising cevimeline hydrochloride hydrate as an active ingredient on saliva secretion in rats. In the graph, “Control” represents an oral mucosa adhesive preparation (not containing cevimeline hydrochloride hydrate but containing a vehicle alone), and “Example” represents an oral mucosa adhesive preparation (administration of 1.25 mg of cevimeline hydrochloride hydrate/rat).

[FIG. 4] A graph showing effects of agents for oral mucosal administration comprising cevimeline hydrochloride hydrate as an active ingredient on saliva secretion in rats. In the graph, “Control” represents an oral mucosa adhesive preparation (not containing cevimeline hydrochloride hydrate but containing a vehicle alone), and “Example” represents an oral mucosa adhesive preparation (administration of 1.5 to 6.0 mg of cevimeline hydrochloride hydrate/rat).

FIG. 5 A graph showing changes with time in concentrations of cevimeline hydrochloride in the rat stomach tissues after oral mucosal administration and oral administration.

FIG. 6 A graph showing changes with time in concentrations of cevimeline hydrochloride in the rat small intestine tissues after oral mucosal administration and oral administration.

FIG. 7 A graph showing changes with time in concentrations of cevimeline hydrochloride in the rat bladder tissues after oral mucosal administration and oral administration.

FIG. 8 A graph showing changes with time in concentrations of cevimeline hydrochloride in the rat kidney tissues after oral mucosal administration and oral administration.

FIG. 9 A graph showing changes with time in concentrations of cevimeline hydrochloride in the rat liver tissues after oral mucosal administration and oral administration.

FIG. 10 A graph showing changes with time in concentrations of cevimeline hydrochloride in the rat plasma after oral mucosal administration and oral administration.

FIG. 11 A graph showing changes with time in concentrations of cevimeline hydrochloride in the rat salivary gland tissues after oral mucosal administration and oral administration.

### BEST MODE FOR CARRYING OUT THE INVENTION

The spirooxathiolane quinuclidine derivatives represented by the aforementioned general formula (I) or acid addition salts thereof are known substances and can be readily obtained by those skilled in the art according to the method disclosed in Japanese Patent Unexamined Patent Publication No. 61-280497. In the general formula (I), examples of the alkyl group include a lower alkyl group having 1 to 6 carbon atoms such as methyl group, ethyl group, propyl group, isopropyl group, n-butyl group, sec-butyl group, tert-butyl group, amyl group and hexyl group. As the aryl group, a substituted or unsubstituted monocyclic or polycyclic aryl group can be used. Examples thereof include phenyl group, tolyl group, xylyl group, diphenyl group, and the like. The monoaryl- or diaryl-substituted methylol group is hydroxymethyl group having 1 or 2 the same or different aryl groups, and the aforementioned aryl groups can be used as the aryl groups. As the aryl-substituted alkyl group, an alkyl group having 1 or 2 the same or different aryl groups can be used, and the aforementioned groups can be used as the alkyl group and the aryl groups. Examples of the aryl-substituted alkyl group include benzyl group, diphenylmethyl group, and the like.

Preferred examples of the spirooxathiolane quinuclidine derivatives include, for example, 2-methylspiro(1,3-oxathiolane-5,3′-quinuclidine), 2-diphenylmethylspiro(1,3-oxathiolane-5,3′-quinuclidine), 2-methyl-2-phenylspiro(1,3-oxathiolane-5,3′-quinuclidine), and the like, and those in the cis-configuration are more preferred. Among them, 2-methylspiro(1,3-oxathiolane-5,3′-quinuclidine) is particularly preferred.

As the spirooxathiolane quinuclidine derivatives, arbitrary geometrical isomers in pure forms, enantiomeric isomers, diastereomers, arbitrary mixtures thereof, racemates thereof, and the like may be used. As the geometrical isomers, the spirooxathiolane quinuclidine derivatives in the cis-configuration are preferably used. 2-Methylspiro(1,3-oxathiolane-5,3′-quinuclidine) in the cis-configuration is a particularly preferred active ingredient. In addition to the use of the compounds in a pure form of cis-configuration as the active ingredient of the medicament of the present invention, mixtures of the compounds in the cis and trans-configurations wherein the compounds in the cis-configuration is contained at a higher ratio can also be used as the active ingredient of the medicament of the present invention.

Examples of the acid addition salts of the spirooxathiolane quinuclidine derivatives include, for example, acid addition salts of inorganic acids or organic acids such as hydrochloric acid, sulfuric acid, phosphoric acid, sulfamic acid, lactic acid, tartaric acid, succinic acid and maleic acid. As the acid addition salt, for example, 2-methylspiro(1,3-oxathiolane-5,3′)quinuclidine hydrochloric acid addition salt is preferred. However, the active ingredient of the medicament of the present invention is not limited to this particular acid addition salt. Arbitrary hydrates or solvates of the spirooxathiolane quinuclidine derivatives or acid addition salts thereof may also be used as the active ingredient of the medicament of the present invention. The most preferred active ingredient is (±)-cis-2-methylspiro(1,3-oxathiolane-5,3′)quinuclidine monohydrochloride hemihydrate (nonproprietary name: “cevimeline hydrochloride hydrate”), and this active ingredient has already been launched in the market as “Evoxac Capsule 30 mg” (xerostomic symptom improving agent for oral administration, produced and sold by Daiichi Pharmaceutical Co., Ltd.).

The form of the medicament of the present invention is not particularly limited so long as a form suitable for oral mucosal administration is chosen, and preferred examples include forms of pharmaceutical compositions of patch, spray, gargle, buccal tablet, sublingual tablet, solution, ointment, jelly, aerosol, film, and the like. The forms of pharmaceutical compositions of patch, gargle, spray and film are more preferred. The state of the medicament of the present invention is not particularly limited, and it may be in an arbitrary state such as solid, semisolid, liquid and gel.

The medicament of the present invention can directly deliver the active ingredient from the oral mucosa to the salivary glands. Since the salivary glands are located at positions very close to the oral mucosa, if the medicament of the present invention is administered by oral mucosal administration, a more efficient and direct saliva secretion promoting effect can be attained compared with the conventional medicaments administered by oral administration. The human salivary glands are roughly classified into two groups, i.e., the major salivary glands (parotid, submandibular and sublingual glands), which are located at positions far from the oral mucosa and connected from the openings at the oral mucosa surface with long ducts, and the minor salivary glands, which exist in extensive areas in the vicinity of the oral mucosa surface such as lips, tongue, palate and cheek. It is considered that much of saliva is usually secreted from the major salivary glands, and only a small amount of saliva is secreted from the minor salivary glands. Since the minor salivary glands have not yet been fully studied, and no method for measuring the exact amount of saliva has been established, many aspects of the minor salivary glands remain unknown. However, it is believed that sensation of dry oral mucosa is highly likely to relate to functions of the minor salivary glands, and saliva secreted from the minor salivary glands plays an important role in the oral mucosa moistening effect.

Since the medicament of the present invention can directly deliver the active ingredient to, in particular, the minor salivary glands locating in the vicinity of the oral mucosa, the medicament can achieve effective prophylactic and/or therapeutic treatment of sensation of dry oral mucosa. Since the distance of oral mucosal diffusion of the active ingredient may be extremely short for the minor salivary glands locating in the vicinity of the oral mucosa, a high drug concentration can be achieved from just after the administration, and high drug efficacy can be expected with a small amount of the active ingredient. Further, a part of the active ingredient can also be expected to directly reach the minor salivary glands via short ducts. Since the minor salivary glands are extensively distributed in the oral cavity, preferred for medicaments targeting the minor salivary glands is the form of spray, gargle, buccal tablet, solution or aerosol, which enables application to extensive areas of the oral mucosa. Spray is a form for ejecting the composition in a liquid form by using air pressure or the like, and aerosol is a form for ejecting the composition in a liquid or powder form by using a propellant such as liquefied gas or compressed gas. By using these measures, powder may be directly ejected to the oral mucosa, or an aqueous composition in the form of solution or suspension may be ejected to the oral mucosa.

The medicament of the present invention is preferably prepared as a pharmaceutical composition containing a water-soluble or swellable polymer and/or a hydrophilic solvent. The water-soluble or swellable polymer means a polymer that is soluble or uniformly dispersed in water and may be swollen by absorption of water upon dissolution or dispersion. This polymer is a substance having an adhesive property to the oral mucosa and plays a role of supplying the active ingredient on the oral mucosa surface when the active ingredient of the medicament of the present invention is absorbed into the oral mucosa.

The type of the water-soluble or swellable polymer is not particularly limited, and, for example, one or more kinds of polymers selected from the group consisting of methylcellulose, ethylcellulose, hydroxyethylcellulose, hydroxymethylethylcellulose, hydroxypropylcellulose, hydroxypropylmethylcellulose, carboxymethylcellulose sodium, xanthan gum, karaya gum, gum arabic, tragacanth gum, cyamoposis gum, gelatin, polyvinylpyrrolidone, polyvinyl alcohol, polyacrylic acid or a metal salt thereof, a carboxyvinyl polymer, an alginic acid salt, propylene glycol alginate, pullulan and a lower alkyl vinyl ether/maleic anhydride copolymer and a derivative thereof may be used. Preferably, for example, one or more kinds of polymers selected from the group consisting of methylcellulose, ethylcellulose, hydroxyethylcellulose, hydroxymethylethylcellulose, hydroxypropylcellulose, hydroxypropylmethylcellulose and carboxymethylcellulose sodium may be used.

As the hydrophilic solvent, an organic solvent miscible with water can be used, and the hydrophilic solvent enhances oral mucosal absorbability of the active ingredient and/or is used as a solubilizing agent of the active ingredient. The type of the hydrophilic solvent is not particularly limited, and, for example, one or more kinds of solvents selected from the group consisting of monoethanolamine, diethanolamine, triethanolamine, diisopropanolamine, triisopropanolamine, ethanol, isopropanol, glycerin, ethylene glycol, propylene glycol, 1,3-butylene glycol, N-methyl-2-pyrrolidone, polyethylene glycol 300, polyethylene glycol 400, triethyl citrate, a glycerin fatty acid ester, polysorbate 20, polysorbate 40, polysorbate 60 and polysorbate 80 may be used. Preferably, one or more kinds of solvents selected from the group consisting of monoethanolamine, diethanolamine, triethanolamine, diisopropanolamine and triisopropanolamine may be used. Triethanolamine can be more preferably used.

The water-soluble or swellable polymer can be used in an amount of about 5 to 80% by weight, preferably 10 to 60% by weight, based on the total weight of the pharmaceutical composition. The hydrophilic solvent can be used in an amount of about 1 to 30% by weight, preferably 5 to 20% by weight, based on the total weight of the pharmaceutical composition. However, it should be understood that the aforementioned amounts can be suitably selected by those skilled in the art.

The medicament of the present invention in the form of a pharmaceutical composition is preferably prepared to have a pH of 4 to 9, preferably 6.5 to 9, more preferably 7 to 8.5. A pharmaceutical composition having such a pH provides particularly favorable oral mucosal absorption of the active ingredient. When pH is higher than 9, the oral mucosal tissues may be degenerated, and irritancy or the like may be caused. The pH of the preparation can be determined by measuring pH of a solution obtained by dissolving or suspending the preparation in water in an amount 3 times the weight of the preparation. For adjusting pH of the pharmaceutical composition, for example, mineral acids such as hydrochloric acid, inorganic compounds such as hydroxides of alkali metals, hydroxides of alkaline earth metals, carbonates and hydrogencarbonates, organic bases such as monoethanolamine, diethanolamine, triethanolamine, diisopropanolamine and triisopropanolamine, organic acids such as methanesulfonic acid, p-toluenesulfonic acid and oxalic acid, and the like may be used as a pH modifier. The pH modifier can be used in a ratio of, for example, about 0.01 to 20% by weight, preferably 0.1 to 15% by weight, of the total weight of the pharmaceutical composition. Two or more kinds of pH modifiers may be suitably used in combination.

Particularly preferred examples of the pharmaceutical composition include a patch comprising (1) a water-insoluble support layer and (2) an adhesive layer showing adhesion to mucosa laminated on the support layer. Oral mucosal administration can be attained by cutting the patch into a suitable size and adhering it to the oral mucosa. The aforementioned adhesive layer has a function of adhering and fixing the pharmaceutical composition to the oral mucosa, and the active ingredient added to this adhesive layer is dissolved out of the adhesive layer and reaches the oral mucosal surface. The active ingredient is penetrated and diffused from the mucosal surface into the mucosa, and reaches to the salivary glands. The water-insoluble support layer has a function of protecting the pharmaceutical composition from the saliva as well as water, drink or food to be contained in the mouth.

The water insoluble support layer may contain, for example, one or more kinds of substances selected from the group consisting of ethylcellulose, hydroxyethylcellulose, hydroxypropylmethylcellulose phthalate, hydroxypropylmethylcellulose acetate succinate, cellulose acetate phthalate, shellac, polyisobutylene and polyisoprene. In addition to the aforementioned form of two laminated layers, the aforementioned pharmaceutical composition may be a pharmaceutical composition comprising three or more laminated layers by adopting two or more layers for either one or both of the aforementioned layers.

In the production of the medicament of the present invention in the form of a pharmaceutical composition, one or more kinds of additives commonly used for pharmaceutical preparations may be used. As the additives for pharmaceutical preparations, for example, one or more kinds of excipients, binders, lubricants, coloring materials, flavoring agents, flavors, surfactants, sweeteners, preservatives, and the like may be used. Active ingredients having other pharmacological activities can also be added.

Examples of the excipients include, for example, anhydrous silicic acid, mannitol, sorbitol, anhydrous calcium phosphate, and the like. Examples of the binders include tragacanth, sodium alginate, and the like. Examples of the lubricants include, for example, magnesium stearate, talc, stearic acid or a salt thereof, a sucrose fatty acid ester, and the like. Examples of the coloring materials include, for example, Blue No. 1, Yellow No. 4, titanium dioxide, and the like. Examples of the flavoring agents include, for example, menthol, peppermint oil, limonene, cineol, citric acid, fumaric acid, tartaric acid etc. as well as various flavors, and the like. Examples of the surfactants include, for example, anionic surfactants, nonionic surfactants, ampholytic surfactants and cationic surfactants, and more specific examples include, for example, sodium laurylsulfate, sucrose fatty acid ester, lactose fatty acid ester, lactitol fatty acid ester, maltitol fatty acid ester, stearic acid monoglyceride, polyoxyethylene sorbitan monolaurate, polyoxyethylene sorbitan monostearate, polyoxyethylene (10, 20, 40, 60, 80, 100 moles) hydrogenated caster oil, polyoxyethylene alkyl ether, polyoxyethylene polyoxypropylene alkyl ester, polyoxyethylene polyoxypropylene alkyl ether, alkylolamide, 2-alkyl-N-carboxymethyl-N-hydroxyethylimidazolinium betaine, and the like. Examples of the sweeteners include, for example, xylitol, erythritol, saccharin sodium, stevioside, stevia extract, paramethoxycinnamic aldehyde, neohesperidyl dihydrochalcone, perilartine, and the like. Examples of the preservatives include, for example, p-hydroxybenzoic acid ester, sodium benzoate, and the like. These additives for pharmaceutical preparations can be used in a ratio of, for example, 0.5 to 10% by weight, preferably 1 to 5% by weight, based on the total weight of the pharmaceutical composition. However, the ratio can be suitably selected by those skilled in the art depending on types of the additives for pharmaceutical preparations or a purpose of addition.

As active ingredients having other pharmacological activities, for example, active ingredients that are effective for prevention of bad breath, periodontal diseases, intraoral diseases, pharyngeal diseases such as throat inflammation, and dental diseases such as dental caries and hypersensitivity, as well as local anesthetics, anti-inflammatory agents, antiphlogistic analgesic agents, and the like may be used, taking into account that xerostomia of Sjogren's syndrome is likely to cause periodontal diseases such as decayed tooth and stomatitis. Further, in addition to the active ingredient contained in the medicament of the present invention, active ingredients that can be expected to have prophylactic and/or therapeutic effects for xerostomia may be added.

### EXAMPLES

The present invention will be explained more specifically with reference to examples. However, the scope of the present invention is not limited by these examples. In the following examples, (±)-cis-2-methylspiro(1,3-oxathiolane-5,3′-quinuclidine) monohydrochloride monohydrate was used as the active ingredient (henceforth this active ingredient is referred to as “cevimeline hydrochloride hydrate,” and (±)-cis-2-methylspiro(1,3-oxathiolane-5,3′-quinuclidine) monohydrochloride as “cevimeline hydrochloride”). The combinations and mixing ratios of the components used in the examples are summarized in the tables. “Gargle (solution type)”

### Examples 1

Cevimeline hydrochloride hydrate was accurately weighed in an amount of 30 mg, added with McIlvaine buffer (McIlvaine buffer: mixture of 0.1 mol/L citric acid and 0.2 mol/L disodium hydrogenphosphate) of pH 8.0 and dissolved to obtain a test solution for gargle in a total volume of 10 mL.

### Example 2

Cevimeline hydrochloride hydrate was accurately weighed in an amount of 30 mg, added with 0.3% aqueous triethanolamine/hydrochloric acid (pH 8.0) and dissolved to obtain a test solution for gargle in a total volume of 10 mL.

### Example 3

Cevimeline hydrochloride hydrate was accurately weighed in an amount of 30 mg, added with 1.5% aqueous triethanolamine/hydrochloric acid (pH 8.0) and dissolved to obtain a test solution for gargle in a total volume of 10 mL.

### Example 4

Cevimeline hydrochloride hydrate was accurately weighed in an amount of 30 mg, added with 10% aqueous ethanol and dissolved to obtain a test solution for gargle in a total volume of 10 mL.

### “Mucosa Adhesive Patch”

### <Preparation Method of Mucosa Adhesive Patch>

### (1) Preparation Method of Support Layer

As the support layer of Comparative Example 1, the components mentioned in Table 1 were mixed in each mixing ratio, and added with an appropriate volume of distilled water, and the mixture was thoroughly stirred to dissolve the components. This solution was uniformly coated on a PET film and dried at 90° C. for 20 minutes to prepare a uniform film having a thickness of about 30 μm. As the support layers of Examples 1 to 6, the components mentioned in Table 1 were mixed in each mixing ratio, and added with an appropriate volume of methylene chloride/ethanol (1:1), and the mixture was thoroughly stirred to dissolve the components. Each solution was uniformly coated on a PET film and dried at 80° C. for 10 minutes to prepare a uniform film having a thickness of 30 to 40 μm.

### (2) Preparation Method of Two-Layer Preparation (See FIG. 1)

The components of the adhesive layer 1) mentioned in Table 1 were uniformly mixed in each mixing ratio, and 20 mg of this mixture was weighed and compression-molded at about 140 kg/cm² for 1 minute by using a KBr tableting machine (Shimadzu Corporation, P/N202-32010, internal diameter of the tableting machine: 13 mm) and a hand press (Shimadzu Corporation, Model SSP-10A, P/N200-64175). This layer was laminated with the support layer having a diameter of 13 mm (thickness: 30 to 40 μm) and compression-molded again at about 140 kg/cm² for 30 seconds. Then, a portion having a diameter of 7 mm was punched out to obtain a test drug for an animal test (thickness: 180 to 200 μm).

### (3) Preparation Method of Three-Layer Preparation (See FIG. 1)

The components of the adhesive layer 1) mentioned in Table 1 were uniformly mixed in each mixing ratio, and 20 mg of this mixture was weighed and compression-molded at about 140 kg/cm² for 1 minute by using a KBr tableting machine (Shimadzu Corporation, P/N202-32010, internal diameter of the tableting machine: 13 mm) and a hand press (Shimadzu Corporation, Model SSP-10A, P/N200-64175). Then a central portion was punched out in a diameter of 5 mm. The components of the adhesive layer 2) were similarly mixed and molded, then a central portion was punched out in a diameter of 5 mm. This punched-out hole was embedded with the adhesive layer 1) having a diameter 5 mm, laminated with a support layer having a diameter of 13 mm (thickness: 30 to 40 μm) thereon and compression-molded again at about 140 kg/cm² for 30 seconds. Then, a portion was punched out in a diameter of 7 mm so that the adhesive layer 1) was positioned at the center to obtain a test preparation for an animal test (thickness: 180 to 200 μm).

### Example 5

As the support layer, 80.0 parts by weight of hydroxypropylmethylcellulose acetate succinate (henceforth abbreviated as “HPMCAS”), 5.0 parts by weight of ethylcellulose and 14.0 parts by weight of polyethylene glycol 400 were added to an appropriate volume of methylene chloride/ethanol (1:1), and dissolved by stirring, 1.0 part by weight of iron sesquioxide was added to the solution and uniformly dispersed, and this mixture was cast on a PET film and dried to prepare a uniform film having a thickness of about 30 μm. Separately, as the adhesive layer 1), 25.1 parts by weight of hydroxypropylmethylcellulose (henceforth abbreviated as “HPMC”), 25.1 parts by weight of polyvinylpyrrolidone, 5.8 parts by weight of polyethylene glycol 400, 1.1 parts by weight of yellow iron sesquioxide, 0.6 parts by weight of sodium hydrogencarbonate and 42.3 parts by weight of cevimeline hydrochloride hydrate were added and uniformly mixed. Further, as the adhesive layer 2), 43.5 parts by weight of HPMC, 43.5 parts by weight of polyvinylpyrrolidone, 10.0 parts by weight of polyethylene glycol 400, 2.0 parts by weight of titanium oxide and 1.0 part by weight of sodium hydrogencarbonate were added and uniformly mixed. These mixtures were molded by compression according to the aforementioned preparation method of three-layer preparation.

### Example 6

As the support layer, 44.0 parts by weight of carboxymethylethylcellulose (henceforth abbreviated as “CMEC”), 44.0 parts by weight of ethylcellulose and 10.0 parts by weight of triethyl citrate were added to an appropriate volume of methylene chloride/ethanol (1:1), and dissolved by stirring, 2.0 parts by weight of yellow iron sesquioxide was added to the solution and uniformly dispersed, and this mixture was cast on a PET film and dried to prepare a uniform film having a thickness of about 30 μm. Separately, as the adhesive layer 1), 25.1 parts by weight of hydroxypropylcellulose, 25.1 parts by weight of polyvinylpyrrolidone, 5.8 parts by weight of polyethylene glycol 400, 1.1 parts by weight of iron sesquioxide, 0.6 part by weight of sodium hydrogencarbonate and 42.3 parts by weight of cevimeline hydrochloride hydrate were added and uniformly mixed. Further, as the adhesive layer 2), 43.5 parts by weight of hydroxypropylcellulose, 43.5 parts by weight of polyvinylpyrrolidone, 10.0 parts by weight of polyethylene glycol 400, 2.0 parts by weight of titanium oxide and 1.0 part by weight of sodium hydrogencarbonate were added and uniformly mixed. These mixtures were molded by compression according to the aforementioned preparation method of three-layer preparation.

### Example 7

As the support layer, 80.0 parts by weight of HPMCAS, 5.0 parts by weight of white shellac and 14.0 parts by weight of polyethylene glycol 400 were added to an appropriate volume of methylene chloride/ethanol (1:1), and dissolved by stirring, 1.0 part by weight of iron sesquioxide was added to the solution and uniformly dispersed, and this mixture was cast on a PET film and dried to prepare a uniform film having a thickness of about 30 μm. Separately, as the adhesive layer 1), 24.2 parts by weight of HPMC, 24.2 parts by weight of polyvinylpyrrolidone, 5.8 parts by weight of polyethylene glycol 400, 1.2 parts by weight of yellow iron sesquioxide, 2.3 parts by weight of sodium hydrogencarbonate and 42.3 parts by weight of cevimeline hydrochloride hydrate were added and uniformly mixed. Further, as the adhesive layer 2), 42.0 parts by weight of HPMC, 42.0 parts by weight of polyvinylpyrrolidone, 10.0 parts by weight of polyethylene glycol 400, 2.0 parts by weight of titanium oxide and 4.0 parts by weight of sodium hydrogencarbonate were added and uniformly mixed. These mixtures were molded by compression according to the aforementioned preparation method of three-layer preparation.

### Example 8

As the support layer, 80.0 parts by weight of HPMCAS, 5.0 parts by weight of ethylcellulose and 14.0 parts by weight of polyethylene glycol 400 were added to an appropriate volume of methylene chloride/ethanol (1:1), and dissolved by stirring, 1.0 part by weight of yellow iron sesquioxide was added to the solution and uniformly dispersed, and this mixture was cast on a PET film and dried to prepare a uniform film having a thickness of about 30 μm. Separately, as the adhesive layer, 35.0 parts by weight of HPMC, 35.0 parts by weight of polyvinylpyrrolidone, 10.8 parts by weight of polyethylene glycol 400, 1.0 part by weight of yellow iron sesquioxide, 1.0 part by weight of triethanolamine and 17.2 parts by weight of cevimeline hydrochloride hydrate were added and uniformly mixed. These mixtures were molded by compression according to the aforementioned preparation method of two-layer preparation.

### Example 9

As the support layer, 80.0 parts by weight of HPMCAS, 5.0 parts by weight of white shellac and 14.0 parts by weight of polyethylene glycol 400 were added to an appropriate volume of methylene chloride/ethanol (1:1), and dissolved by stirring, 1.0 part by weight of iron sesquioxide was added to the solution and uniformly dispersed, and this mixture was cast on a PET film and dried to prepare a uniform film having a thickness of about 30 μm. Separately, as the adhesive layer, 26.0 parts by weight of HPMC, 26.0 parts by weight of polyvinylpyrrolidone, 10.5 parts by weight of polyethylene glycol 400, 1.0 part by weight of titanium oxide, 2.0 parts by weight of triethanolamine and 34.5 parts by weight of cevimeline hydrochloride hydrate were added and uniformly mixed. These were molded by compression according to the aforementioned preparation method of two-layer preparation.

### Example 10

As the support layer, 80.0 parts by weight of HPMCAS, 5.0 parts by weight of ethylcellulose and 14.0 parts by weight of polyethylene glycol 400 were added to an appropriate volume of methylene chloride/ethanol (1:1), and dissolved by stirring, 1.0 part by weight of yellow iron sesquioxide was added to the solution and uniformly dispersed, and this mixture was cast on a PET film and dried to prepare a uniform film having a thickness of about 30 μm. Separately, as the adhesive layer, 8.0 parts by weight of HPMC, 8.0 parts by weight of polyvinylpyrrolidone, 10.0 parts by weight of polyethylene glycol 400, 1.0 part by weight of yellow iron sesquioxide, 4.0 parts by weight of triethanolamine and 69.0 parts by weight of cevimeline hydrochloride hydrate were added and uniformly mixed. These were molded by compression according to the aforementioned preparation method of two-layer preparation.

### Example 11

As the support layer, 46.0 parts by weight of CMEC, 38.0 parts by weight of ethylcellulose and 15.0 parts by weight of triethyl citrate were added to an appropriate volume of methylene chloride/ethanol (1:1), and dissolved by stirring, 1.0 part by weight of yellow iron sesquioxide was added to the solution and uniformly dispersed, and this mixture was cast on a PET film and dried to prepare a uniform film having a thickness of about 30 μm. Separately, as the adhesive layer 1), 25.1 parts by weight of HPMC, 25.1 parts by weight of polyvinylpyrrolidone, 5.8 parts by weight of polyethylene glycol 400, 1.1 parts by weight of yellow iron sesquioxide, 0.6 part by weight of triethanolamine and 42.3 parts by weight of cevimeline hydrochloride hydrate were added and uniformly mixed. Further, as the adhesive layer 2), 43.5 parts by weight of HPMC, 43.5 parts by weight of polyvinylpyrrolidone, 10.0 parts by weight of polyethylene glycol 400, 2.0 parts by weight of titanium oxide and 1.0 part by weight of triethanolamine were added and uniformly mixed. These mixtures were molded by compression according to the aforementioned preparation method of three-layer preparation.

### Control

As a support layer, 80.0 parts by weight of hydroxyethylcellulose, 10.0 parts by weight of polyvinylpyrrolidone and 9.0 parts by weight of polyethylene glycol 400 were added to an appropriate volume of distilled water, and dissolved by stirring, 1.0 part by weight of yellow iron sesquioxide was added to the solution and uniformly dispersed, and this mixture was cast on a PET film and dried to prepare a uniform film having a thickness of about 30 μm. Separately, as an adhesive layer, 44.5 parts by weight of hydroxypropylcellulose, 44.5 parts by weight of polyvinylpyrrolidone, 10.0 parts by weight of polyethylene glycol 400 and 1.0 part by weight of sodium hydrogencarbonate were added and uniformly mixed. These mixtures were molded by compression according to the aforementioned preparation method of two-layer preparation.

TABLE 1

| | | Examples | | | | | | | | |
|---|---|---|---|---|---|---|---|---|---|---|
| Layer | Component | Control | 5 | 6 | 7 | 8 | 9 | 10 | 11 | |
| Support | HPMCAS | — | 80.0 | — | 80.0 | 80.0 | 80.0 | 80.0 | — | |
| layer | CMEC | — | — | 44.0 | — | — | — | — | 46.0 | |
| | Ethylcellulose | — | 5.0 | 44.0 | — | 5.0 | — | 5.0 | 38.0 | |
| | White shellac | — | — | — | 5.0 | — | 5.0 | — | — | |
| | Hydroxyethylcellulose | 80.0 | — | — | — | — | — | — | — | |
| | Polyvinylpyrrolidone | 10.0 | — | — | — | — | — | — | — | |
| | Triethyl citrate | — | — | 10.0 | — | — | — | — | 15.0 | |
| | Polyethylene glycol 400 | 9.0 | 14.0 | — | 14.0 | 14.0 | 14.0 | 14.0 | — | |
| | Yellow iron sesquioxide | — | — | 2.0 | — | — | — | — | 1.0 | |
| | (coloring agent) | | | | | | | | | |
| | Iron sesquioxide | 1.0 | 1.0 | — | 1.0 | 1.0 | 1.0 | 1.0 | — | (w/w %) |
| | (coloring agent) | | | | | | | | | |
| Adhesive | Hydroxypropylcellulose | 44.5 | — | 43.5 | — | — | — | — | — | |
| layer (2) | HPMC | — | 43.5 | — | 42.0 | 35.0 | 26.0 | 8.0 | 43.5 | |
| | Polyvinylpyrrolidone | 44.5 | 43.5 | 43.5 | 42.0 | 35.0 | 26.0 | 8.0 | 43.5 | |
| | Polyethylene glycol 400 | 10.0 | 10.0 | 10.0 | 10.0 | 10.8 | 10.5 | 10.0 | 10.0 | |
| | Titanium oxide | — | 2.0 | 2.0 | 2.0 | — | 1.0 | — | 2.0 | |
| | Yellow iron sesquioxide | — | — | — | — | 1.0 | — | 1.0 | — | |
| | Sodium | 1.0 | 1.0 | 1.0 | 4.0 | — | — | — | — | |
| | hydrogencarbonate | | | | | | | | | |
| | Triethanolamine | — | — | — | — | 1.0 | 2.0 | 4.0 | 1.0 | |
| | Cevimeline | — | — | — | — | 17.2 | 34.5 | 69.0 | — | (w/w %) |
| | hydrochloride hydrate | | | | | | | | | |
| Adhesive | Hydroxypropylcellulose | | — | 25.1 | — | | | | — | |
| layer (1) | HPMC | | 25.1 | — | 24.2 | | | | 25.1 | |
| | Polyvinylpyrrolidone | | 25.1 | 25.1 | 24.2 | | | | 25.1 | |
| | Polyethylene glycol 400 | | 5.8 | 5.8 | 5.8 | | | | 5.8 | |
| | Iron sesquioxide | | — | 1.1 | — | | | | — | |
| | Yellow iron sesquioxide | | 1.1 | — | 1.2 | | | | 1.1 | |
| | Sodium | | 0.6 | 0.6 | 2.3 | | | | — | |
| | hydrogencarbonate | | | | | | | | | |
| | Triethanolamine | | — | — | — | | | | 0.6 | |
| | Cevimeline | | 42.3 | 42.3 | 42.3 | | | | 42.3 | (w/w %) |
| | hydrochloride hydrate | | | | | | | | | |

HPMCAS: Hydroxypropylmethylcellulose acetate succinate
CMEC: Carboxymethylethylcellulose
HPMC: Hydroxypropylmethylcellulose

### Test Example 1

### Influences of pH and Additives on Drug Absorption from Human Oral Mucosa

Healthy volunteers were made to clean their teeth and then gargle with 10 mL of each test solution (Examples 1 to 4) for 2 minutes, the gargles were collected, and then the oral cavity was washed twice with 10 mL of distilled water for 10 seconds. All the mouth wash was added to the test solution after gargling. The amounts of cevimeline hydrochloride in the test solutions before and after the gargling were measured by HPLC, and the difference between the samples was calculated as the amount of absorption to the oral mucosa.

Influences of pH and additives on oral mucosal absorption of cevimeline hydrochloride hydrate are shown in FIG. 2. At pH 8.0, a larger absorption amount was obtained with triethanolamine/hydrochloric acid buffer compared with that obtained with McIlvaine buffer (mixture of 0.1 mol/L citric acid and 0.2 mol/L disodium hydrogenphosphate), and oral mucosal absorbability was better at the triethanolamine concentration of 1.5% than at 0.3%. These results suggested that triethanolamine had an effect of promoting mucosal absorption of cevimeline hydrochloride hydrate. In addition, more cevimeline hydrochloride hydrate was absorbed at pH 8.0 than at pH 6.0.

### Test Example 2

### Promotion of Saliva Secretion in Rats (Oral Mucosal Administration)

Rats (12-week old male Wistar rats, body weight: 195 to 240 g) were fixed at the dorsal position under urethane (1.25 g/kg i.p.) anesthesia. Prior to the start of the test, saliva in the oral cavity was thoroughly wiped off with a paper wiper (trade name: Kimwipe) beforehand, then saliva in the oral cavity was adsorbed again 15 minutes later (time point 0) by using three piece of paper wiper (weighed beforehand), which were cut into a size of 35×30 mm and rolled. Immediately after this procedure, each test preparation (Examples 5, 6, 8 to 10, Control) was administered, and then saliva was adsorbed by the same procedure every 15 minutes to calculate the amount of saliva secretion. After completion of the test, the condition of the application site of the preparation was observed. The amount of saliva secretion was calculated as follows:

Saliva amount (mg)=(Paper wiper weight after adsorption of saliva)−(Paper wiper weight before adsorption of saliva)

Effects of agents for oral mucosal administration of cevimeline hydrochloride hydrate on saliva secretion in rats are shown in FIGS. 3 and 4. The oral mucosa adhesive preparations of Examples 5 and 6 containing 1.25 mg of cevimeline hydrochloride hydrate tended to increase the amount of saliva secretion from the time points of 45 and 60 minutes after the administration, respectively. Further, the oral mucosa adhesive preparations of Examples 9 (containing 3.0 mg) and 10 (containing 6.0 mg) increased the amount of saliva secretion in rats from the time points of 15 and 30 minutes after the treatment, respectively. Further, a clear difference was recognized between the preparations in Examples 9 (containing 3.0 mg) and 10 (containing 6.0 mg) in the saliva secretion promoting effect. When the condition of the oral mucosa tissues at the application site was macroscopically observed after completion of the test, no irritancy was observed.

### Test Example 3

### Drug Distribution to Each Tissue in Rats (Comparison of Oral Mucosal Administration and Oral Administration)

Rats (9-week old male Wistar rats, body weight: 172 to 205 g) were fixed at the dorsal position under urethane (1.25 g/kg i.p.) anesthesia. The test preparation was administered, and plasma and tissues (minor salivary glands, stomach, small intestine, liver, kidney) were collected 0.5, 1, 2, 4 and 8 hours later for observation of the condition of the application site of the preparation and measurement of the concentration in each tissue. The surface of the minor salivary glands was rinsed with physiological saline, and contents were removed from the stomach and the small intestine, which were rinsed in physiological saline. Each collected tissue was stored at −40° C. before the measurement. The medicament concentration in each tissue was measured by LC/MS/MS. The oral mucosal patch of Example 9 was used for the test, and the results were compared with those obtained by oral administration.

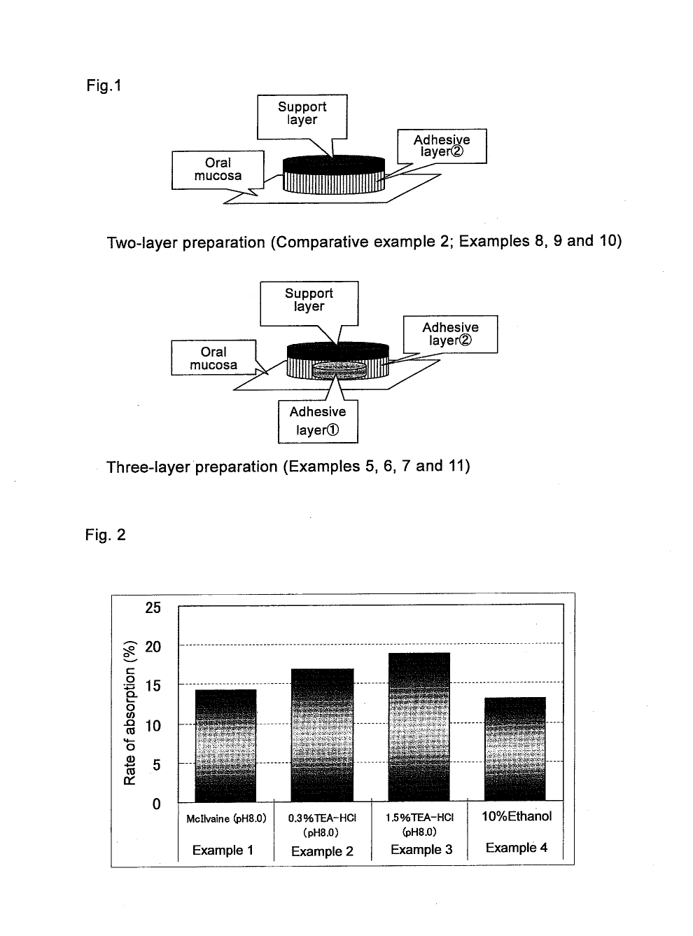

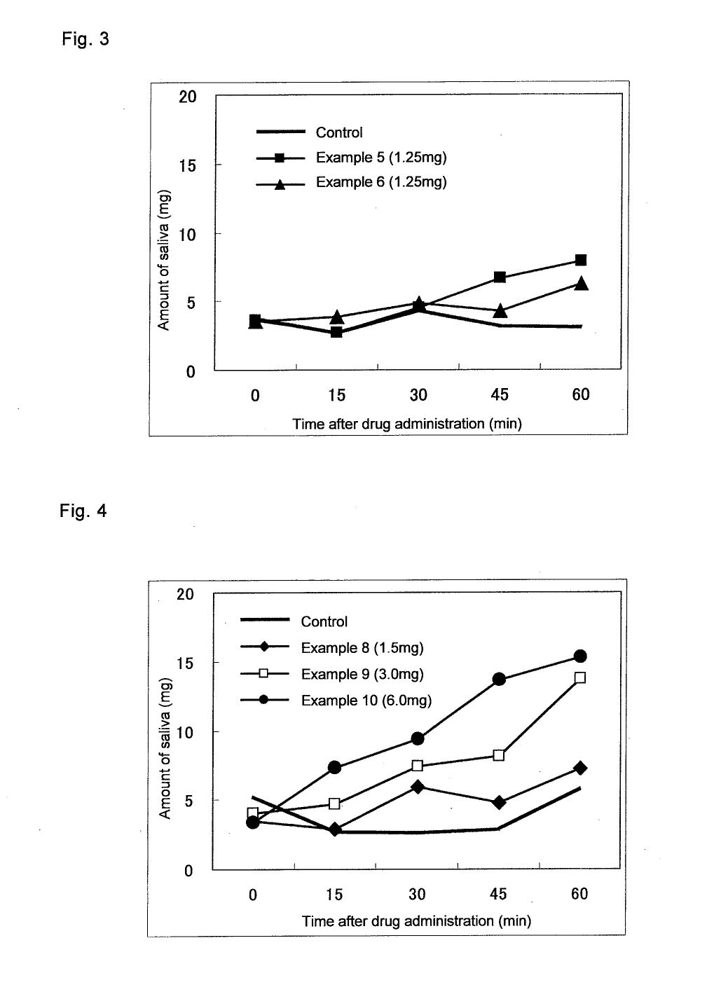

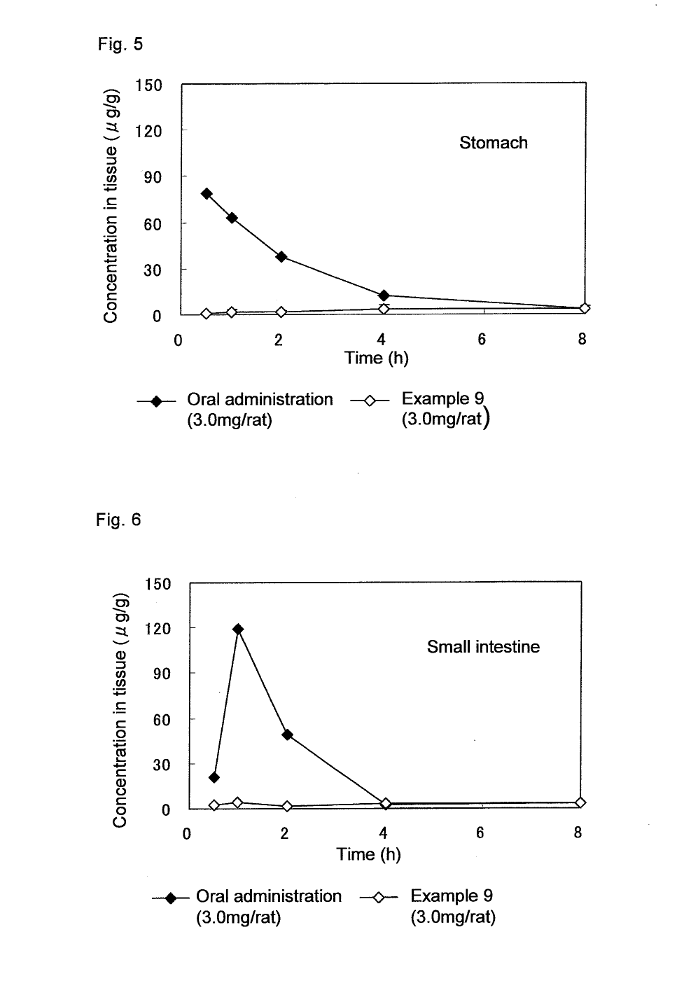

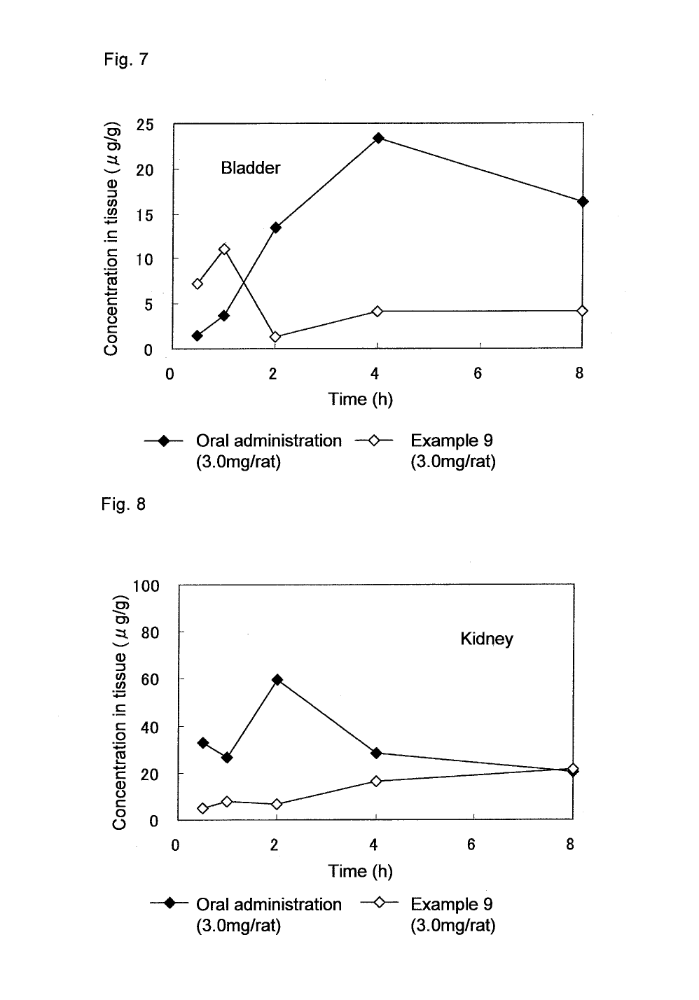

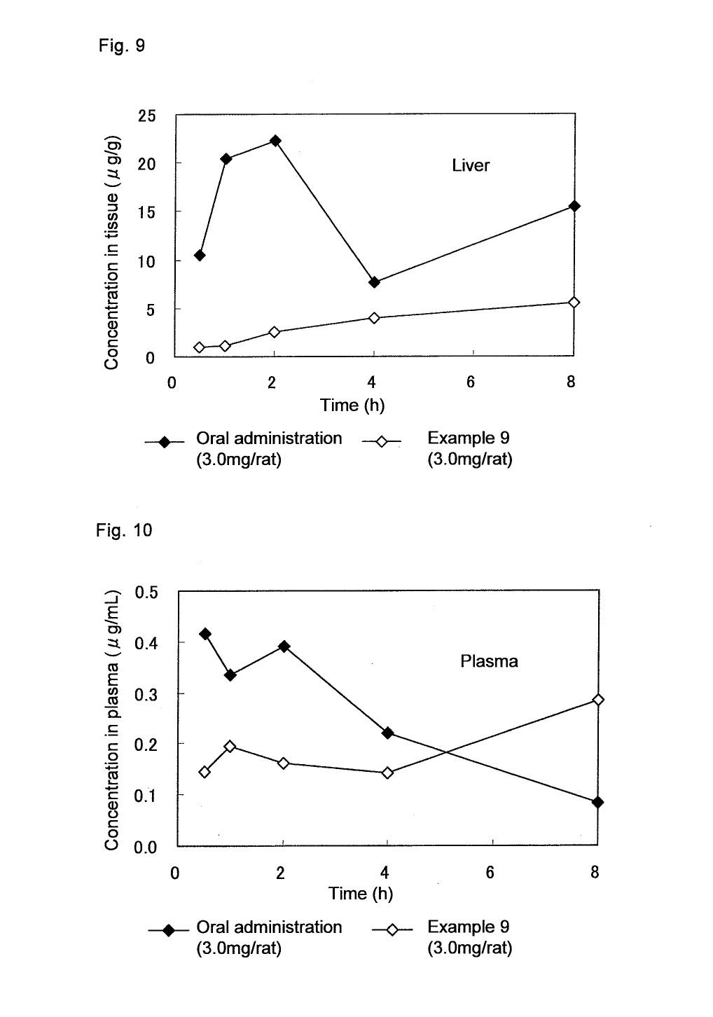

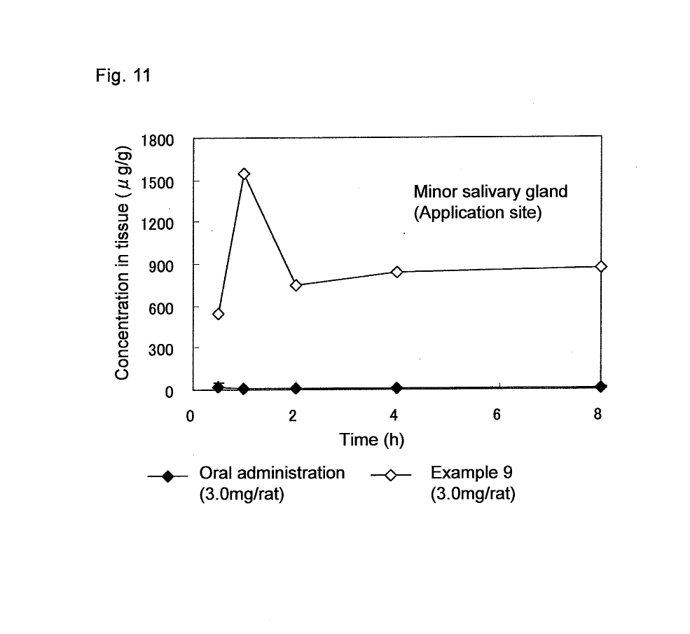

Changes with time in the cevimeline hydrochloride hydrate concentration in each tissue of the rats after the oral administration and the oral mucosal administration are shown in FIGS. 5 to 11. After the oral mucosal administration, the drug concentrations in the stomach, small intestine, kidney and liver were lower and showed less change compared with those obtained by the oral administration. In particular, the medicament concentrations in the stomach, small intestine, kidney and liver were apparently low in the initial period after administration (up to 2 hours). Further, the plasma concentrations changed less in the initial period after the administration than after the oral administration and remained constantly low thereafter. These results suggested that oral mucosal administration possibly prevented development of adverse reactions in the digestive system (nausea, abdominal pain, diarrhea, pollakiuria and the like), which are considered to occur due to the high concentrations in the gastrointestinal tract tissues. Further, since oral mucosal administration achieved markedly high concentrations in the minor salivary glands as the target sites thereof, it was considered that the medicament directly reached to the site from the oral mucosa and acted. The above results revealed that cevimeline hydrochloride hydrate had higher absorbability from the oral mucosa, and that the medicament administered by oral mucosal administration more directly acted on the target sites compared with the same medicament administered by oral administration. Further, concentrations in the digestive system tissues were low and no mucosal irritancy was observed, and accordingly, oral mucosal administration was found to be an administration method superior in safety. For the oral mucosal treatment group, a possibility was considered that the medicament was swallowed and absorbed from the gastrointestinal tract in addition to absorption from the oral mucosa. However, the amount of the medicament distributed to the gastrointestinal tract after the administration of the oral mucosa adhesive preparation was much lower than that observed after the oral administration, and therefore, it was presumed that influences of swallowing of the oral mucosa adhesive preparation was negligible.

### Preparation Example 1

### Film Preparation

A preparation for oral mucosal administration containing cevimeline hydrochloride hydrate in the form of film, paper or medicinal wafer is a thin film enabling absorption of medicament from the oral mucosa by extensively releasing the drug in the oral cavity and adhering the drug to the oral mucosa. Such a preparation for oral mucosal administration is described in, for example, International Patent Unexamined Publication in Japanese (KOHYO) Nos. 2001-506612 and 2001-506640, and those skilled in the art can easily produce the aforementioned preparation by referring to these publications. The entire disclosures of the publications are incorporated by reference as the disclosure of the present specification.

TABLE 2

| | Example | | |
|---|---|---|---|
| Component | 12 | 13 | 14 |
| Cevimeline hydrochloride hydrate | 17.0 | 30.0 | 12.0 |
| Polyvinylpyrrolidone | 35.0 | — | — |
| Polyvinyl alcohol | — | 25.5 | — |
| Hydroxypropylmethylcellulose | — | — | 30.0 |
| Starch | 35.0 | — | — |
| Titanium oxide | 5.0 | 5.0 | — |
| Silicon oxide | — | 20.0 | — |
| Polysorbate 80 | — | — | 1.0 |
| Brij35 | — | — | 35.0 |
| Flavor | 2.0 | 2.5 | 5.0 |
| Sorbitol | 2.0 | 3.0 | 5.0 |
| Aspartame | — | — | 3.0 |
| pH modifier | 4.0 | 4.0 | 3.0 |
| 85% Glycerin | — | 10.0 | 6.0 |
| Ethanol | 30.0 | — | 250.0 |
| Distilled water | 100.0 | 100.0 | 250.0 |

Composition (g)

### Preparation Example 2

### Ointment and Jelly

Jelly and ointment containing cevimeline hydrochloride hydrate are semisolid preparations enabling drug absorption from the oral mucosa on which they are applied, and they can be prepared, for example, according to the following prescriptions.

TABLE 3

| | Example | | | | | |
|---|---|---|---|---|---|---|
| Component | 15 | 16 | 17 | 18 | 19 | 20 |
| Cevimeline hydrochloride | 10.0 | 10.0 | 10.0 | 10.0 | 10.0 | 10.0 |
| hydrate | | | | | | |
| White Vaseline | 50.0 | — | — | — | 25.0 | — |
| Plastibase | 10.0 | — | — | — | — | — |
| Pectin | 15.0 | — | — | — | — | — |
| Polyvinyl alcohol | 5.0 | — | — | — | — | — |
| Carboxymethylcellulose Na | — | 10.0 | — | — | — | — |
| Polyethylene glycol 4000 | — | — | — | 36.0 | — | — |
| Cetanol | — | — | 10.0 | — | — | 4.0 |
| Stearyl alcohol | — | — | — | — | 20.0 | 6.0 |
| Polyethylene glycol 400 | — | — | — | 54.0 | — | — |
| Liquid paraffin | — | — | 15.0 | — | — | 3.0 |
| Glycerin | — | — | 20.0 | — | — | — |
| Propylene glycol | — | — | — | — | 12.0 | 30.0 |
| Polyoxyethylene sorbitan fatty | — | — | 5.0 | — | | — |
| acid ester | | | | | | |
| Polyoxyethylene (60) | — | — | — | — | 4.0 | — |
| hydrogenated caster oil | | | | | | |
| Glyceryl monostearate | — | — | — | — | 1.0 | — |
| Polyoxyl stearate 40 | — | — | — | — | — | 5.0 |
| Sorbitan sesquioleate | — | — | — | — | — | 2.0 |
| Methyl paraoxybenzoate | — | 0.1 | — | — | 0.1 | — |
| Propyl paraoxybenzoate | — | 0.1 | — | — | 0.1 | — |
| Flavor | 1.0 | 1.0 | 0.5 | — | — | — |
| Sorbitol | 6.0 | 10.0 | — | — | — | — |
| Saccharin sodium | — | — | 0.5 | — | — | — |
| PH modifier | 3.0 | 4.0 | 3.0 | — | 3.0 | 3.0 |
| Distilled water | — | 64.8 | 36.0 | — | 24.8 | 37.0 |

Composition (g)

### Preparation Example 3

### Aerosol

An aerosol filled in a spray can with a propellant so that a solution or a suspension containing cevimeline hydrochloride hydrate can be ejected is a preparation enabling application of the drug to extensive areas of the oral mucosa and drug absorption from the oral mucosa. The aerosol can be prepared, for example, according to the following prescriptions.

TABLE 4

| | Example | | | | | |
|---|---|---|---|---|---|---|
| Component | 21 | 22 | 23 | 24 | 25 | 26 |
| Cevimeline hydrochloride | 10.0 | 10.0 | 10.0 | 10.0 | 10.0 | 10.0 |
| hydrate | | | | | | |
| Isopropanol | — | — | — | — | 5.0 | — |
| Propylene glycol | — | — | — | — | — | 10.0 |
| Glycerin | — | — | — | 10.0 | — | — |
| Polyoxyethylene (60) | 5.0 | 5.0 | 5.0 | 5.0 | 5.0 | 5.0 |
| hydrogenated caster oil | | | | | | |
| Benzalkonium chloride | — | — | 0.01 | — | — | — |
| Methyl paraoxybenzoate | 0.1 | 0.1 | — | — | — | — |
| Propyl paraoxybenzoate | 0.03 | — | — | — | — | — |
| Sorbitol | — | 5.0 | — | — | — | — |
| Lactose | — | — | — | 5.0 | — | — |
| pH modifier | 3.0 | 3.0 | 3.0 | 3.0 | 3.0 | 3.0 |
| Ethanol | 5.0 | 5.0 | 5.0 | — | — | — |
| Distilled water | 76.87 | 71.9 | 76.99 | 67.0 | 77.0 | 72.0 |

Composition (g)

### Preparation Example 4

### Gargle (Water-Soluble Polymer Containing Type)

A gargle in the form of a solution containing cevimeline hydrochloride hydrate or in the form of powder used by being dissolved with an appropriate volume of a solution is a preparation that can be applied to extensive areas of the oral mucosa and enables drug absorption from the oral mucosa. The preparation can be manufactured, for example, according to the following prescriptions. As for a gargle in the form of powder, 150 mg of cevimeline hydrochloride hydrate is dissolved by adding 20 to 150 mL of water, and gargling is performed one to five times per day for 1 to 5 minutes with this solution or suspension contained in the mouth.

TABLE 5

| | Gargle (solution) | | | Gargle (powder) | | |
|---|---|---|---|---|---|---|
| | Example | | | Example | | |
| Component | 27 | 28 | 29 | 30 | 31 | 32 |
| Cevimeline hydrochloride hydrate | 150 | 150 | 150 | 150 | 150 | 150 |
| Hydroxypropylmethylcellulose | 200 | — | — | — | — | — |
| Polyvinylpyrrolidone | — | 200 | — | 100 | — | — |
| Pullulan | — | — | 200 | — | 150 | — |
| Xanthan gum | — | — | — | — | — | 150 |
| Lactose | — | — | — | 200 | — | — |
| Sucrose | 640 | — | — | 530 | — | — |
| Sorbitol | — | 640 | — | — | 690 | — |
| Xylitol | — | — | 647.5 | — | — | 687.5 |
| Triethanolamine | 10 | — | — | 10 | — | — |
| Diisopropanolamine | — | 10 | — | — | 10 | — |
| Sodium hydrogencarbonate | — | — | 2.5 | — | — | 2.5 |
| Anhydrous calcium chloride | — | — | — | 10 | — | 10 |
| Distilled water | 100,000 | 100,000 | 100,000 | | | |

Composition (mg)

### INDUSTRIAL APPLICABILITY

The medicament of the present invention allows extremely quick distribution of a spirooxathiolane quinuclidine derivative or an acid addition salt thereof as an active ingredient from the oral mucosa into the salivary glands, and exhibits a superior saliva secretion promoting effect from just after the administration. Further, the medicament of the present invention can remarkably decrease distribution of the active ingredient into the gastrointestinal tract by achieving absorption of the active ingredient from the oral mucosa, and reduce or eliminate adverse reactions in the digestive organs which are problems of conventional oral agents.

1. A medicament in the form for oral mucosal administration comprising a spirooxathiolane quinuclidine derivative represented by the following general formula (I) or an acid addition salt thereof: 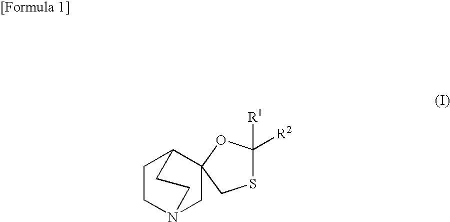
(wherein R¹ and R² may be the same or different, and independently represent a hydrogen atom, an alkyl group, a cyclopentyl group, a cyclohexyl group, a monoaryl- or diaryl-substituted methylol group, or an aryl-substituted alkyl group) as an active ingredient.

2. The medicament according to claim 1, which is for prophylactic and/or therapeutic treatment of xerostomia.

3. The medicament according to claim 1, wherein the spirooxathiolane quinuclidine derivative is 2-methylspiro(1,3-oxathiolane-5,3′-quinuclidine)

4. The medicament according to claim 1, wherein the spirooxathiolane quinuclidine derivative is cis-2-methylspiro(1,3-oxathiolane-5,3′-quinuclidine).

5. The medicament according to claim 1, wherein the active ingredient is 2-methylspiro(1,3-oxathiolane-5,3′-quinuclidine) hydrochloride or a hydrate thereof.

6. The medicament according to claim 1, wherein the active ingredient is cis-2-methylspiro(1,3-oxathiolane-5,3′-quinuclidine) monohydrochloride hemihydrate.

7. The medicament according to claim 1, which is in the form of a pharmaceutical composition containing a water-soluble or swellable polymer and/or a hydrophilic solvent.

8. The medicament according to claim 7, wherein the water-soluble or swellable polymer consists of one or more kinds of polymers selected from the group consisting of methylcellulose, ethylcellulose, hydroxyethylcellulose, hydroxymethylethylcellulose, hydroxypropylcellulose, hydroxypropylmethylcellulose, carboxymethylcellulose sodium, xanthan gum, karaya gum, gum arabic, tragacanth gum, cyamoposis gum, gelatin, polyvinylpyrrolidone, polyvinyl alcohol, polyacrylic acid or a metal salt thereof, a carboxyvinyl polymer, an alginic acid salt, propylene glycol alginate, pullulan, a lower alkyl vinyl ether/maleic anhydride copolymer and a derivative thereof.

9. The medicament according to claim 7, wherein the water-soluble or swellable polymer consists of one or more kinds of polymers selected from the group consisting of methylcellulose, ethylcellulose, hydroxyethylcellulose, hydroxymethylethylcellulose, hydroxypropylcellulose, hydroxypropylmethylcellulose and carboxymethylcellulose sodium.

10. The medicament according to claim 7, wherein the hydrophilic solvent consists of one or more kinds of solvents selected from the group consisting of monoethanolamine, diethanolamine, triethanolamine, diisopropanolamine, triisopropanolamine, ethanol, isopropanol, glycerin, ethylene glycol, propylene glycol, 1,3-butylene glycol, N-methyl-2pyrrolidone, polyethylene glycol 300, polyethylene glycol 400, triethyl citrate, a glycerin fatty acid ester, polysorbate 20, polysorbate 40, polysorbate 60 and polysorbate 80.

11. The medicament according to claim 7, wherein the hydrophilic solvent consists of one or more kinds of solvents selected from the group consisting of monoethanolamine, diethanolamine, triethanolamine, diisopropanolamine and triisopropanolamine.

12. The medicament according to claim 1, wherein pH of the pharmaceutical composition is in the range of 6.5 to 9.

13. The medicament according to claim 1, which is in the form of a pharmaceutical composition of patch, sublingual tablet, buccal tablet, solution, gargle, spray, aerosol, ointment, jelly or film.

14. The medicament according to claim 1, which is in the form of an oral mucosal patch comprising (1) a water-insoluble support layer and (2) an adhesive layer showing adhesion to mucosa laminated on the support layer.

15. The medicament according to claim 14, wherein the water-insoluble support layer contains one or more kinds of substances selected from the group consisting of ethylcellulose, hydroxyethylcellulose, hydroxypropylmethylcellulose phthalate, hydroxypropylmethylcellulose acetate succinate, cellulose acetate phthalate, shellac, polyisobutylene and polyisoprene, and the mucosa adhesive layer contain a water-soluble or swellable polymer that develops adhesion with moisture in the oral cavity, and a hydrophilic solvent.
